(19) United States
(12) Patent Application Publication
(10) Pub. No.: US 2020/0205707 A1
(43) Pub. Date: Jul. 2, 2020

(54) WEARABLE SENSOR FOR ACQUISITION OF BIOMETRICS DATA

(71) Applicant: Think Biosolution Limited, Dublin (IE)

(72) Inventors: Shourjya Sanyal, Tuam (IE); Koushik Kumar Nundy, Dublin (IE)

(73) Assignee: Think Biosolution Limited, Dublin (IE)

(21) Appl. No.: 16/232,182

(22) Filed: Dec. 26, 2018

(51) Int. Cl. A61B 5/1455; A61B 5/00; A61B 5/103

(52) U.S. Cl. CPC A61B 5/14551; A61B 5/6823; A61B 5/1032; A61B 5/742

(57) ABSTRACT

A method for photoplethysmography measurement couples a color camera in optical contact against a skin surface of a subject and couples at least a first solid-state illumination source in optical contact against the skin surface, wherein the first solid-state illumination source has a first wavelength range with a first bandwidth that exceeds 50 nm. The illumination source and color camera are energized over a predetermined time interval to acquire a first sequence of image frames from the skin surface. A set of hue values is computed from each of the acquired sequence of image frames and photoplethysmography data generated according to periodic changes in an average hue per frame computation. The generated photoplethysmography data is presented on a display.

### FIELD OF THE INVENTION

This disclosure generally relates to biometrics and more particularly relates to an apparatus and methods for non-invasive contact based biometric acquisition using wearable color photoplethysmography (PPG) devices.

### BACKGROUND OF THE INVENTION

There is significant interest in development and use of real-time health and wellness tracking devices that acquire and report biometric data for a range of health care and athletic development applications. Various wearable devices have been marketed or proposed for use in measurement and reporting of biometrics such as activity level, body temperature, pulse rate (or heart rate) and heart rate variability, respiratory rate, blood pressure, levels of blood sugar, oxygen saturation, and other physical or chemical indicators related to bodily function.

Photoplethysmography (PPG) applies optical principles for non-invasive measurement of biometrics using reflected or refracted light from subdermal tissue. The PPG signal that is obtained can be used, for example, to readily detect blood volume changes in the subdermal tissue as well as to sense other, subtler effects. PPG measurement is characterized as having a continuously varying (“AC”) waveform that is indicative of synchronous changes in the blood volume with each heartbeat, superimposed on a slowly varying (“DC”) signal baseline that has lower frequency components indicative of respiration, sympathetic nervous system activity, thermoregulation, and other dynamically changing biometric data.

Conventional devices for non-invasive measurement of PPG and other biometrics have proven utility in a number of applications, such as pre and post-operative care, and ambulatory care. However, there are considered to be a number of shortcomings with existing solutions for PPG acquisition. Conventional solutions can be bulky, restricting their use as wearable instruments. Devices that clamp to the finger can be effective for periodic use, but are not practical for applications where movement is a factor or where continuous monitoring is needed during strenuous activity. Measurement methods for PPG have shown reasonable accuracy, but there is felt to be considerable room for improvement.

Thus, it can be seen that there is a need for improved apparatus for non-invasive wearable biometrics.

### SUMMARY OF THE INVENTION

It is an object of the present disclosure to advance the art of biometrics acquisition and analysis. With this object in mind, the present disclosure provides apparatus and methods for obtaining accurate, real-time information on biometric and health information of a subject without the need for obtrusive instrumentation.

According to an embodiment of the present disclosure, there is provided a method for photoplethysmography measurement comprising:
a) coupling a color camera in optical contact against a skin surface of a subject;
b) coupling at least a first solid-state illumination source in optical contact against the skin surface, wherein the first solid-state illumination source has a first wavelength range with a first bandwidth that exceeds 50 nm;
c) energizing the illumination source and color camera over a predetermined time interval to acquire a first sequence of a plurality of image frames from the skin surface;
d) computing a set of hue values from each of the acquired sequence of image frames and generating photoplethysmography data according to periodic changes in an average hue per frame computation; and
e) presenting the generated photoplethysmography data on a display.

These and other aspects, objects, features and advantages of the present invention will be more clearly understood and appreciated from a review of the following detailed description of the preferred embodiments and appended claims, and by reference to the accompanying drawings.

### BRIEF DESCRIPTION OF THE DRAWINGS

While the specification concludes with claims particularly pointing out and distinctly claiming the subject matter of the present disclosure, it is believed that the invention will be better understood from the following description when taken in conjunction with the accompanying drawings.

FIG. 1 is a graph showing aspects of PPG signal content that can be acquired by biometrics sensor apparatus according to an embodiment of the present disclosure.

FIG. 2A is a graph showing measurement parameters when using two PPG signals obtained simultaneously.

FIG. 2B is a graph showing the differences in light absorption as expressed by the absorption coefficients over visible and infrared ranges.

FIG. 3 is a schematic block diagram showing an exemplary apparatus for a biometrics sensor apparatus for contact sensing of biometrics according to an embodiment of the present disclosure.

FIGS. 4A and 4B show two exemplary arrangements of a biometrics sensor apparatus according to embodiments of the present disclosure.

FIG. 5 is a schematic diagram that shows an arrangement of illumination and sensing components at the skin interface for a biometrics sensor apparatus.

FIG. 6 is a logic flow diagram showing an overall processing sequence for contact acquisition and processing of biometric data according to an embodiment of the present disclosure.

FIG. 7 is a logic flow diagram showing processing logic for calculations from a PPG that is obtained using only a single color channel.

FIG. 8 is a logic flow diagram showing processing logic for calculations using PPG data that is obtained using both visible and an IR wavelengths.

FIG. 9 is a timing diagram that shows synchronization of IR and Red illumination used to acquire PPG data used for SpO₂ measurement.

### DETAILED DESCRIPTION OF THE INVENTION

The present description is directed in particular to elements forming part of, or cooperating directly with, apparatus in accordance with the invention. It is to be understood that elements not specifically shown or described may take various forms well known to those skilled in the art.

Figures shown and described herein are provided in order to illustrate key principles of operation and component relationships according to the present invention and are not drawn with intent to show actual size or scale. Some exaggeration may be necessary in order to emphasize basic structural relationships or principles of operation or simply in order to fit components within the available space on the page. Some conventional components that would be needed for implementation of the described embodiments, such as various types of connectors or mounts, for example, may not be shown in the drawings in order to simplify description of the invention itself. In the drawings and text that follow, like components are designated with like reference numerals, and similar descriptions concerning components and arrangement or interaction of components already described may be omitted.

Where they are used, the terms “first”, “second”, and so on, do not denote any ordinal or priority relation, but are simply used to more clearly distinguish one element from another.

In the context of the present disclosure, the term “subject” refers to the person who is being imaged and, in optical terms, can be considered equivalent to the “object” of the corresponding imaging system. The subject can be a patient, an athlete, or other person from whom the biometric data is acquired.

The term “set”, as used herein, refers to a non-empty set, as the concept of a collection of elements or members of a set is widely understood in elementary mathematics. The terms “subset” or “partial subset”, unless otherwise explicitly stated, are used herein to refer to a non-empty proper subset, that is, to a subset of the larger set, having one or more members. For a set S, a subset may comprise the complete set S. A “proper subset” of set S, however, is strictly contained in set S and excludes at least one member of set S. A “partition of a set” is a grouping of the set's elements into non-empty subsets so that every element is included in one and only one of the subsets. Two sets are “disjoint” when they have no element in common.

With respect to an image detector, the term “pixel” refers to a picture element unit cell containing a photo-conversion circuit and related circuitry for converting incident electromagnetic radiation to an electrical signal.

The term “biometric” can be used as an adjective, such as for “biometric data”, or a noun, such as in “acquiring a biometric”.

In the context of the present disclosure, the phrase “optical contact” has its meaning as understood in the optical arts. Two optical surfaces in optical contact can be in physical contact, having no air between them, or at least insufficient air between them to allow reflection between the surfaces to be observed or to allow interference fringes to be formed. Use of a light pipe and, optionally, a suitable index-matching fluid can provide sufficient optical contact between illumination components and the skin or between sensor components and the skin.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

### Biometric Parameters of Interest

Pulse/heart rate (HR) is defined as the number of times the heart beats per minute. Heart rate variability (HRV) is defined as the amount of variance between each consecutive heartbeat. HR is widely used in the assessment of cardiac health such as predicting myocardial infarctions, and HRV in the study of the autonomic nervous system. In sports medicine, for example, HR combined with speed is used to compute predictors like heart rate-running speed index, which can then be used to track, and therefore improve, the cardiac endurance of an athlete. HRV as a function of time is used to determine if an athlete is overtraining.

Both HR and HRV can be accurately measured using electrocardiography (ECG) which uses an array of electrodes on the skin surface to track electrical changes arising from the heart muscles' electrophysiologic pattern of depolarizing and repolarizing during each heartbeat. However, the multiple-lead ECG is an impractical instrument for portability, compactness, robustness, and other factors that relate to wearability for a subject patient in motion. HR and HRV can also be reliably measured using photoplethysmography, wherein the variation of transmissivity and/or reflectivity of light is measured through the tissue as a function of arterial pulsation, followed by signal post-processing.

Respiratory rate (RR) is quantified in terms of number of breaths per minute. In clinical settings, RR is widely used as an indicator of respiratory dysfunction and overall circulatory health. For patients, RR can be measured using capnography, that analyzes changes in concentration or partial pressure of carbon dioxide in exhaled gases.

Alternately, pneumography or spirometry which measures change of pressure can be employed for RR measurement. In sports medicine, RR can be used to help distinguish between aerobic and anaerobic regimens. Under practical conditions of intense athletic activity such as rapid subject movement, RR proves to be difficult to measure using most conventional methods.

Blood oxygen saturation (SpO₂) is a measure of the percentage of oxygen that is bound to hemoglobin in peripheral blood. In clinical applications, SpO₂ levels are used to monitor circulatory conditions such as anemia. In sports medicine, SpO₂ combined with running speed is used to track and therefore improve predictors such as vVO2max, velocity at maximal oxygen uptake. SpO₂ is measured using the ratios of the varying transmissivity of alternating pulses of infrared and red light on the skin surface, due to the differential transmissivity of oxyhemoglobin (HbO₂) and hemoglobin (Hb) at varying wavelengths.

Biometric Parameters Obtained from PPG Signal

A brief overview of photoplethysmography (PPG) signals and their information content is instructive for an understanding of the Applicant's approach to PPG signal acquisition and measurement. As noted previously, photoplethysmography (PPG) applies optical principles for non-invasive measurement of circulation and oxygenation cycles using reflected or refracted light from skin tissue. In the context of the present disclosure, the terms “PPG” and “PPG signal” or “PPG curve” are generally synonymous.

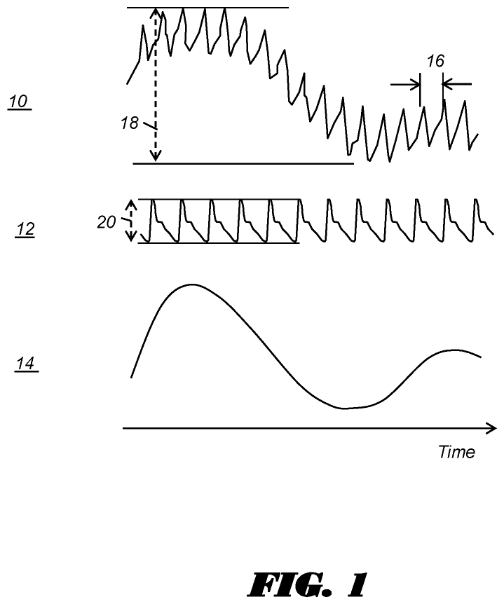

As shown in FIG. 1, a PPG signal 10, shown as a curve generated from individual readings of PPG data, has a continuously varying (“AC”) waveform that is indicative of synchronous changes in the blood volume with each heartbeat. The higher-frequency AC waveform is superimposed on a slowly varying (“DC”) signal baseline that has lower frequency components indicative of respiration, sympathetic nervous system activity, thermoregulation, and other dynamically changing biometric data. A single continuous PPG measurement is capable of providing the data needed for HR, HRV, and RR measurement.

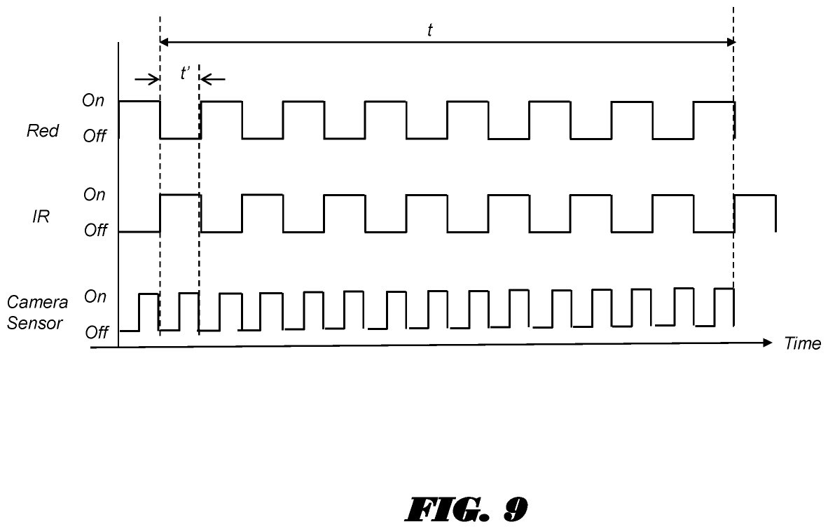

The graph of FIG. 1 shows aspects of the PPG signal content that can be acquired by biometrics sensor apparatus. Continuous PPG signals can be obtained by using a single set of continuously energized LEDs. Emission wavelengths can be in the visible or infrared range, for example. The raw or unprocessed PPG signal 10 has both higher and lower frequencies, as shown over a time interval. A high-pass filtered PPG signal 12 allows more accurate analysis of various stages in the circulatory cycle. A low-pass filtered PPG signal 14 can then show respiratory rate. Among signal features of interest is peak-to-peak distance 16 for measuring HRV. Signal amplitudes 18 and 20 are useful in subsequent computation. Simultaneous measurement of paired PPG signals can be used to measure blood oxygenation, based on the differential absorption of oxygenated hemoglobin (oxyhemoglobin, HbO₂) and non-oxygenated hemoglobin (Hb). Paired PPG signals can be obtained by using two sets of alternatively energized LEDs, wherein each set of LEDs has a different range of emission wavelengths (a visible emission set and an infrared (IR) emission set). The LEDs are alternatively energized, in synchronization with the optical sensor, such that alternating temporal data corresponds to visible and IR emission wavelengths. Timing for alternating signals is described subsequently with reference to FIG. 9.

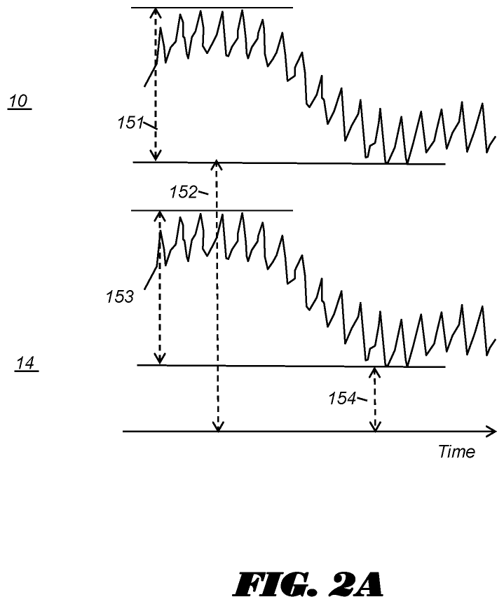

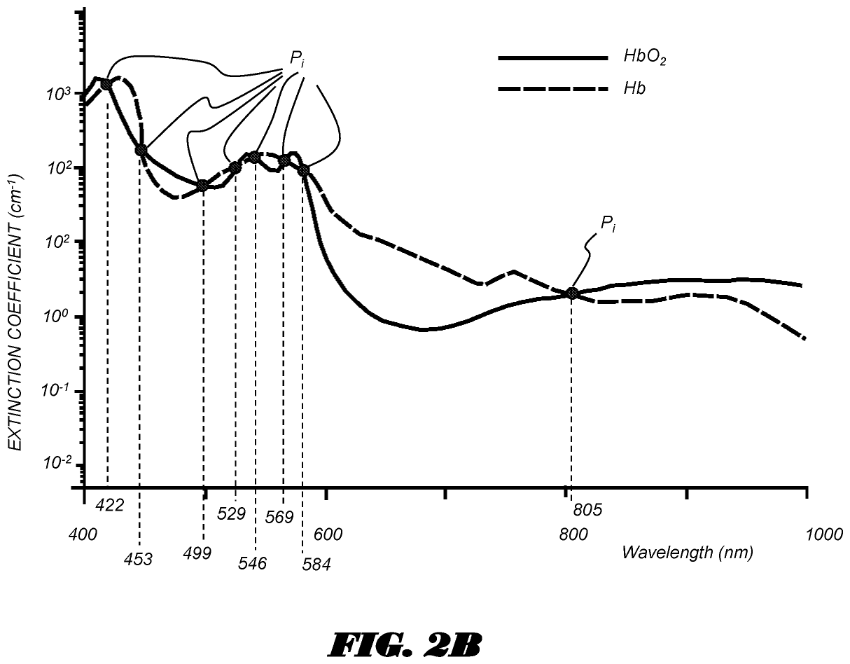

As shown in FIG. 2A, for PPG signal 10 corresponding to the red emission wavelength range, the ratio of the signal amplitudes 151 to basal intensity 152 gives a quantity termed Red_{AC/DC}.

Another ratio applies for IR light. For PPG signal 14 corresponding to the IR emission wavelength range, the ratio of signal amplitudes 153 to basal intensity 154 gives a quantity termed IR_{AC/DC}.

The ratio of these two ratios yields a useful quantity for SpO₂ computation:

$$R = \frac{{Red}_{AC \text{/} DC}}{{IR}_{AC \text{/} DC}}$$

SpO₂ in percentage can be derived using the relation:

SpO₂=25*R+110

(Values 25 and 110 are constants, empirically determined.)

The visible and IR wavelengths used for SpO₂ measurement are carefully selected by considering the extinction coefficients of oxyhemoglobin HbO₂ and hemoglobin Hb as shown in the graph of FIG. 2B. Of particular note in the FIG. 2B graph is the observation that, for light over some wavelength regions, the Hb extinction coefficient (dashed line in FIG. 2B) is higher than the corresponding coefficient for HbO₂; for light over other wavelength regions, the HbO₂ extinction coefficient is higher than the corresponding coefficient for Hb. Significantly, FIG. 2B highlights isosbestic points Pᵢ, wavelengths at which the two extinction coefficients are equal. Some representative isosbestic points Pᵢexist at wavelengths 453 nm, 499 nm, 529 nm, 569 nm, and others, where the respective spectra curves for Hb and HbO₂ cross each other. In embodiments of the present disclosure, isosbestic points Pᵢ define the boundaries of the chosen range of emission wavelengths, so that the emitted wavelengths are within ranges defined and bounded between adjacent “nearest-neighbor” isosbestic points Pᵢ. The wavelength ranges used for calculation in the Applicant's embodiments intentionally avoid including any isosbestic values, for reasons described in detail subsequently. In the context of the present disclosure, two isosbestic points are considered to be adjacent isosbestic points if there is no intermediate isosbestic point among the wavelengths between them.

There can be benefits and advantages to wearable devices for biometrics acquisition that address known problems with existing biometrics solutions and are improved with respect to a number of criteria, including:
(i) Wearable and easily attached to or integrated with athletic, occupational, recreational, or hospital attire. Ease of wearability is related to factors such as device weight, size, and overall ergonomics.
(ii) Unobstructive, allowing natural movement of the subject, including normal motion. This can include movement at rest as well as motion during strenuous exertion or during athletic activity. This attribute can also mean wearing of the measurement device continuously.
(iii) Accurate, offering improved measurement results when compared against existing devices. Accuracy can relate to factors such as high signal-to-noise ratio, for example. Optical contact between the sensor and skin is highly desirable.
(iv) Robust, able to provide useful measurements even during subject movement.
(v) Insensitive to factors such as skin coloration, perspiration, patient weight.
(vi) Multi-featured, able to provide biometric data related to multiple circulatory functions, unlike biometrics solutions that measure only heart rate or only oxygenation. PPG can be measured most accurately from particular portions of the anatomy, such as the upper chest (sternum). At this position, for example, PPG measurements can provide additional information on respiratory rate.

Conventional PPG measurement devices have been shown to be workable and provide accuracy, but fail to provide a solution that meets demanding criteria for robustness and that is easily wearable and usable in diverse environments. Embodiments of the present disclosure provide biometric measurement based on color PPG measurement that addresses criteria for practical usability such as those listed above. The Applicant's approach measures color fluctuation using a single broadband color component, as described subsequently.

### Modes of Operation

There are two modes of measurement operation for apparatus of the present disclosure:
1. Single PPG Mode—PPG measurement from continuous illumination is capable of providing the data needed for HR, HRV, and RR measurement. A “continuously illuminated” mode can be obtained using continuous illumination from (i) a broadband Red LED, (ii) a broadband IR LED, or (iii) both a broadband red and broadband IR LED, wherein by “broadband” is meant emitting over a range with bandwidth larger than 50 nm and smaller than about 100 nm.
2. Dual PPG Mode—Simultaneous measurement and comparison of two paired PPG signals can be used to measure blood oxygenation SpO₂. The two PPG signals can be measured using alternating Red and IR LED illumination at 15 frames per second, with a camera recording at 30 frames per second, for example.

Contact Approach

A number of PPG measurement apparatus employ a video camera or other imaging sensing device aimed toward the subject that measures changes in the skin of the subject, using a set of acquired image frames. Sensing of the skin color variation is performed in “non-contact” mode, that is, with the optical sensing spaced apart from the skin surface. In order to obtain improved performance, the Applicant has developed camera-based apparatus that have illumination and imaging light paths continuously in optical contact with the skin of the subject for acquiring PPG measurement. This optical contact approach necessitates a number of changes to the design of the camera or scanner that obtains the PPG image content, as well as to approaches used for illumination of the skin surface. For example, the illumination is provided as diffused light, refracted through the subdermal tissue rather than reflected from the skin, as in the non-contact video signal of conventional designs. In order to obtain the desired improvement, optical coupling must be maintained with the subject's skin surface during movement, including under periods of intense subject activity and exertion.

Among advantages of the Applicant's approach are compactness in packaging, efficient use of illumination even where diffused, and capability to acquire image content directly from the skin of the subject in spite of vigorous physical activity. The Applicant's approach is suited to a number of embodiments and physical arrangements, including integration with headgear, gloves, and other articles of clothing.

### Apparatus

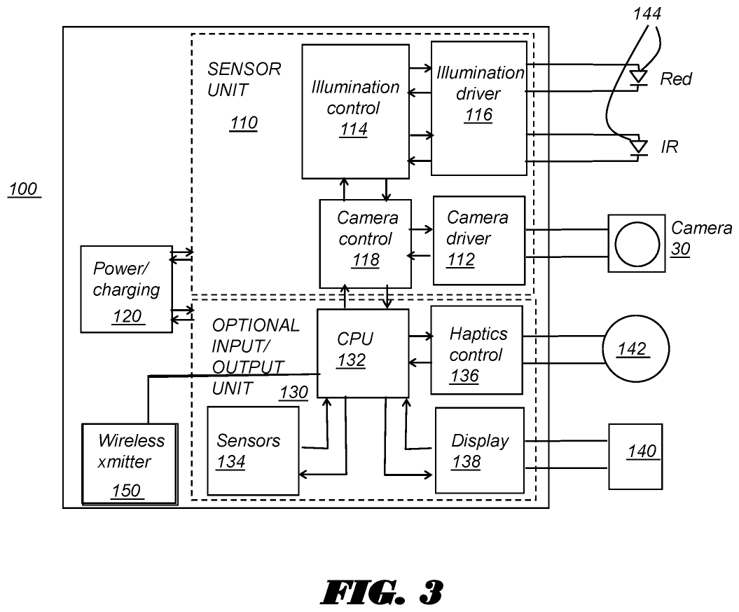

The schematic of FIG. 3 shows an exemplary apparatus for a biometrics sensor apparatus 100 for contact sensing of biometrics according to an embodiment of the present disclosure. Sensor apparatus 100 has a sensor unit 110, a power and charging circuit 120, and an optional input/output (I/O) unit 130. Sensor unit 110 can include a camera controller 118 and camera driver 112 that is in signal communication with a camera 30 or other light sensor for acquiring image frames, an illumination control 114, and an illumination driver 116 that can control any of a number of types of LED 144 or other illumination source. I/O unit 130 can have a control logic processor or CPU 132, optional haptics controller and driver 136 that can connect to a haptics device 142 for haptic feedback, for example, a display unit 138 that is in signal communication with an integral or external display 140, and a sensor unit 134 that can include additional sensing components such as an accelerometer, gyroscope, and magnetometer, for example. Devices capable of providing multi-dimensional movement sensing can include a GPS module sensing position, for example. Still other supported biometric tracking devices can include a single- or multiple-lead ECG or a temperature sensor.

According to an embodiment of the present disclosure, camera 30 is a dedicated sensor device that is designed specifically for contact imaging. Camera 30 acquires a sequence of image frames from its field of view, providing these acquired image frames to processing logic that generates the resulting PPG data according to image frame content.

A wireless transmitter 150 provides the acquired data to a remote display or other device, such as a smartphone, tablet, laptop computer, or other external device for optional display, storage, and transmission. Alternately, a wired transmission option can be provided.

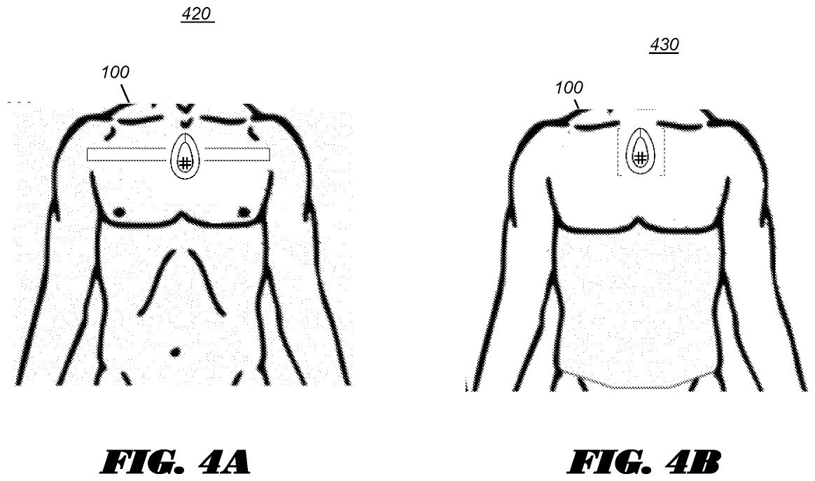

FIGS. 4A and 4B shows two exemplary arrangements of biometrics sensor apparatus 100 according to embodiments of the present disclosure. According to an embodiment 420 sensor apparatus 100 is held against the sternum of the user by a chest strap 200. An embodiment 430 shows apparatus 100 positioned by a shirt or other clothing, such as by form-fitting athletic apparel. The illumination sources and camera can be held in optical contact against the skin in both 420 and 430 embodiments.

The illumination sources and camera are individually parameterized for embodiments 420 and 430. For example, different amounts of pressure may be applied against the skin surface depending on the physical arrangement that supports the camera 30 and corresponding illumination apparatus. A vest-mounted sensor apparatus 100 may require different measurement parameters than those required with a strap-mounted apparatus 100.

It should be noted that apparatus 100 can be configured for skin contact along other portions of the anatomy, such as for measurements from the hand, forehead, or other area. Appropriate changes must be made for maintaining optical contact during PPG measurement at other locations.

### Illumination and Sensing Hardware for PPG Signal Acquisition

Contact-based acquisition of PPG data presents a number of difficulties for portable detector design. In order to obtain accurate color information, illumination and sensing optics must address problems specific to sensing light that is highly diffused through the subject's skin.

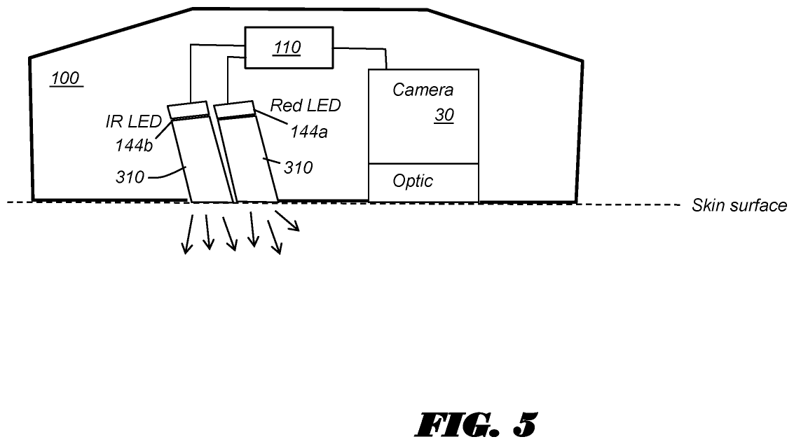

The schematic diagram of FIG. 5 shows an arrangement of illumination and sensing components at the skin interface for biometrics sensor apparatus 100. Significant considerations for illumination include the following:
(i) One or two broadband LED bandwidths can be used as illuminants. There can be one or multiple LEDs of a given bandwidth. FIG. 5 shows an embodiment with illumination from two LEDs having two different bandwidths: an LED 144b provides infrared (IR) light, with broadband emission over the range of wavelengths greater than 700 nm; another LED 144a emits broadband visible light in the Red region, between 620 and 700 nm. Each of the LEDs, in their respective bandwidths has at least a 50 nm broadband emission range, to provide sufficient variation for accurately measuring changes in hue. To provide sufficient accuracy for measuring two color PPG signals and subsequently SpO₂, the red or IR broadband LED emission source should be between two isosbestic points shown in FIG. 2B, such that for the IR, the extinction coefficients of HbO₂ exceed those of Hb, and for Red, the extinction coefficients of Hb exceed those of HbO₂. Avoidance of isosbestic values helps to minimize ambiguity in measurement. Also, there should be no timing overlap during emission of Red and IR illumination sources.
(ii) For measuring HR, RR, and HRV, continuous emission of either LED 144a or LED 144b is sufficient. This emission pattern constitutes Single PPG Mode, in which continuous emission with corresponding image frame capture repeated at appropriate intervals yields a single-color (or IR) PPG signal. For measuring SpO₂ emission, Dual PPG Mode is used. In Dual PPG operation, alternation between the two LEDs 144a and 144b, in a continuing cycle between visible and IR illumination, provides two color PPG signals that can be compared and used for computation.
(iii) LED brightness. Increased LED 144a, 144b brightness is needed in order to provide sufficient light through the subdermal tissue and to the camera 30 sensor. This requirement must be balanced with considerations for LED surface temperatures that allow comfortable skin contact. Separate brightness optimization can be used for IR and visible light LEDs.
(iv) Angle illumination. Characteristics of the illuminating LED 144 include having a large emission angles; for example, with a half angle +/−55-60°.
(v) Optimized LED distance to camera. For each type of illumination (visible or IR) and for use along different parts of the body, separate LED-to-camera distance optimization can be applied (Example: 12.5 mm for chest). As the LED-to-camera distance increases, current through the LED must increase in order to generate sufficient brightness; this can cause heat levels uncomfortable for the subject. As the LED-to-camera distance decreases, insufficient light may be directed through tissue, decreasing the signal-to-noise ratio.

Optical contact of each illuminating LED against the skin surface is maintained by a light guide 310. Light guide 310 can be formed from an optical polycarbonate or from optical liquid silicone rubbers (LSRs), or optical urethanes and polyurethanes, or from a clear epoxy, such as ADHERE™ Opti-tec 5012 Clear Epoxy Adhesive/Encapsulant, an optical epoxy from Intertronics, Oxfordshire, U.K. Optical polycarbonates can be relatively poor as heat conductors and can provide total internal reflection (TIR) for directing light within apparatus 100.

Camera considerations include the following:
(i) Video stream. According to an embodiment of the present disclosure, the camera acquires image frames in a video stream (more than 10 frames/second), for a limited time duration (20 seconds).
(ii) Polychromatic. The camera is required to sense wavelengths emitted from the LED light sources and effectively has three 2D arrays of Red, Green, and Blue sensors. The camera should not have an IR filter.

### Signals Obtained

The signal obtained by the color camera 30 is a succession of images, each a composite array of Red, Green, and Blue (RGB) 2D signals.

A conventional approach for PPG measurement when using a conventional pulse-oximeter employs a single photodiode or an array of photodiodes that measure intensity of the incident light over the green wavelength range. This is equivalent to using measurements from the Green color 2D array of the color camera 30 employed by non-contact camera based methods.

However, the Applicant has found that measuring the PPG signal from the Green 2D array falls short of needed accuracy for PPG measurement. Instead, improved accuracy can be obtained by conversion of the color data to the Hue, Saturation, Value (HSV) color model and using the Hue value. According to an embodiment of the present disclosure, a PPG signal from single-color data, computed using an averaged time-series, is used to form an image in Hue 2D array space, that is, a 2D array of Hue values that is representative of image content and color.

The Applicant has found that a single color PPG curve computed using an averaged time-series of Hue 2D arrays provides a more robust and useful measure of biometric parameters such as HR, RR, HRV relative to conventional methods. Similarly, in Dual PPG mode, as noted previously, two overlapping color PPG signals, computed using two averaged time-series of Hue 2D arrays, provide a more robust and useful measure of biometric parameters such as SpO₂, compared to conventional approaches.

Processing Sequence—Single-Color PPG Mode

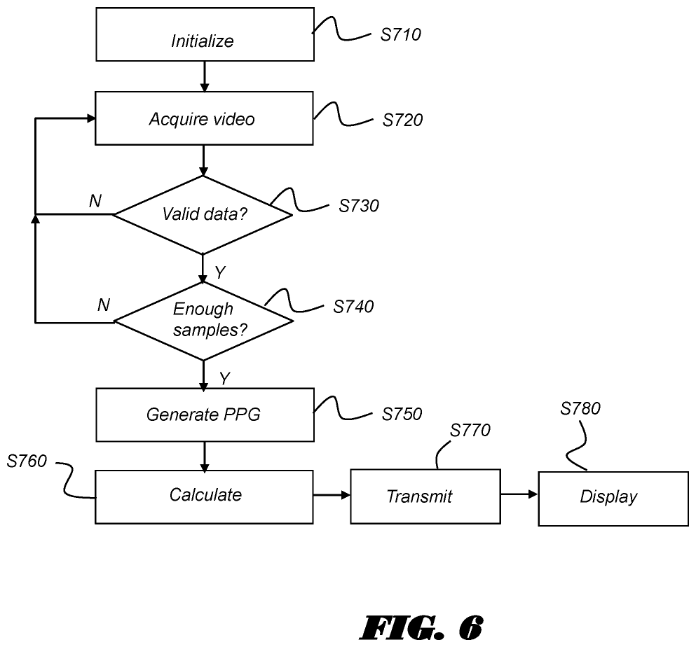

The logic flow diagram of FIG. 6 shows the overall processing sequence for contact acquisition and processing of biometric data according to an embodiment of the present disclosure. These general steps apply for PPG signal generation using either or both visible Red channel and IR channels.

An initialization step S710 begins the sequence, resetting counters and registers for acquiring the biometric data. An acquisition step S720 acquires a sequence of video frames from camera 30 for a given period, such as for about 20 seconds at 10 or more frames per second.

A check step S730 determines whether or not the acquired data is valid or should be discarded. For example, data values may be ambiguous or there may be considerable noise content in the received signal.

A check step S740 determines whether or not sufficient valid samples have been collected for the calculations that follow and repeats frame acquisition as needed.

A PPG signal generation step S750 generates one or more PPG signals that provide a basis for subsequent biometric data calculation, according to periodic changes in the average hue per frame computation. The average hue per frame computation simply calculates hue values from each frame and generates an average value.

A calculation step S760, described in more detail subsequently, calculates the biometric results using the generated PPG data.

An optional transmit step S770 transmits the biometric results for display in a display step S780. The data can be stored and used for subsequent comparison and further calculation.

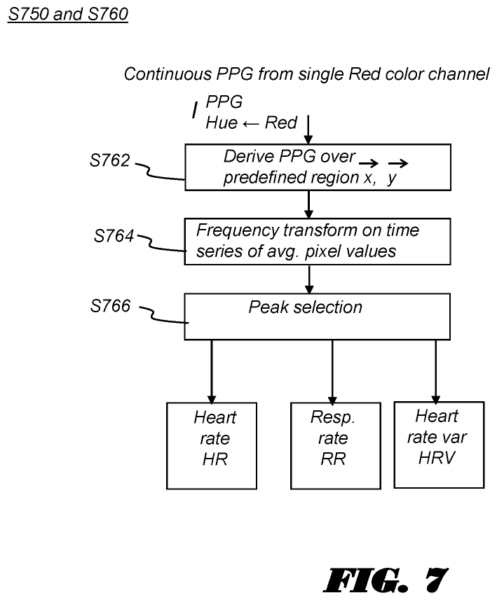

The logic flow diagram of FIG. 7 shows a sequence used for signal generation step S750 and calculation step 760 when using a single red LED illumination according to an embodiment of the present disclosure. In a derivation step S762, the following value can be derived from the sensed PPG signal content:

I_{Hue←Red}^{PPG}=ΣₜΣ_{\vec{x},\vec{y}}I_{Hue(Hue,Saturation,Value)}

A single color PPG is computed as the average of the Hue channel I_{Hue(Hue,Saturation,Value)} for frames numbered 0 to t. For each frame, the averaging is done over a predefined region of pixels (this 2D space is referenced as \vec{x} and \vec{y}). Value I_{Hue(Hue,Saturation,Value)} for each pixel in this region is derived from I_{Red,Green,Blue} using the corresponding R,G, B values and standard RGB-to-HSV conversion formulas that are known to those skilled in the imaging arts.

Continuing with the FIG. 7 sequence, a transform step S764 does a frequency transform of the single PPG, such as using a Fast Fourier Transform (FFT) or other method. In a peak selection step S766, biometric parameters for HR, HRV, and RR values can be subsequently computed using a peak selection algorithm and applying an appropriate band-pass filter. This sequence can be followed by application of HR bandpass filters corresponding to the frequency range of interest, typically associated with HR values; exemplary 3 dB cutoff values are at 0.8 to 2.2 Hz. The frequency range for RR values has 3 dB cutoff at about 0.18 to 0.5 Hz. According to an embodiment, the order for the HR filter is 20; the order for the RR filter is 8.

Computation is illustrated in FIG. 1. The peaks of the filtered frequency spectra I_{Hue←Red}^{PPG}f(HR) and I_{Hue←Red}^{PPG}f(RR) correspond to HR and RR readings, respectively. To visualize the effect of the filter on the raw PPG signal, it is replotted as shown at 12 and 14 in FIG. 1. HRV can be computed from step S764 results relating to the width of the I_{Hue←Red}^{PPG}f(HR) peak, or, equivalently, by measuring the peak-to-peak distance in the raw signal I_{Hue←Red}^{PPG}.

The color PPG signal can be written as:

I_{Hue←Red}^{PPG}=ΣₜΣ_{λ}Σ_{\vec{x},\vec{y}}\hat{P}(λ)h(λ,\vec{x},\vec{y},t)×[v_{DC}(\vec{x},\vec{y},t)b_{DC}(λ,t)+v_{AC}(\vec{x},\vec{y},t)b_{AC}(λ,t)]

wherein \hat{P}(λ) is the power of a given illumination source at a given wavelength λ, and wherein ĥ(λ) indicates the CIE (International Commission on Illumination) color-matching functions that account for the response of the eye, camera, or other optical sensor.

By using the Hue channel, instead of the green channel, embodiments of the present disclosure allow measurement of fluctuation along the wavelength axis, instead of the absorption coefficient axis.

The observable PPG signal can be expressed as a function of pulsatile (AC) and non-pulsatile (DC) components:

$${I}_{G}^{PPG} = {\sum }_{t} I ( \vec{x} , \vec{y} , t ) {\sum }_{λ} {\sum }_{\vec{x} + \vec{y}} \hat{P } ( λ ) h ( λ , \vec{x} , \vec{y} , t ) [ {v}_{DC} ( \vec{x} , \vec{y} , t ) {b}_{DC} ( λ , t ) + {v}_{AC} ( \vec{x} , \vec{y} , t ) {b}_{AC} ( λ , t ) ]$$

The time-dependent variance of the pulsatile (AC) component (absent in the color PPG) is strongly correlated to the ECG (electrocardiogram) signal, allowing measurement of HR, HRV and RR values.

As noted previously, while processing for the visible Red signal is used as an example for measurement using Hue, IR illumination could also be used for single-color illumination, with similar processing.

### Processing Sequence—Dual PPG Mode

As noted previously, alternating illumination with Red and IR LEDs enables accurate measurement of blood oxygenation SpO₂. SpO₂ levels are measured indirectly, by calculating the ratio of averaged hemoglobin concentration to averaged total concentration of hemoglobin in the blood.

$${SpO}_{2} = \frac{〈 {HbO}_{2} 〉}{〈 {HbO}_{2} 〉 + 〈 Hb 〉}$$

According to an embodiment of the present disclosure, the needed values are obtained from a ratio of ratios of two PPGs derived from Red (λ₁) and IR (λ₂) sources, using a linear regression curve:

SpO₂=β₁R_{λ}_{₁}_{,λ}_{₂}+α₁

Wherein both the y-intercept β₁ and the slope α₁ are empirically obtained. Typical values: β₁˜−25; α₁˜110.

The basic logic flow of FIG. 6 also applies for dual PPG mode, with necessary changes for handling the alternating signal content. For example, initialization and acquisition steps S710 and S720 perform many of the same basic steps and the data validation checks of steps S730 and S740 are similarly executed.

The two alternating input color PPGs are I_{Hue←Red}^{PPG} and I_{Hue←IR}^{PPG}:

$${I}_{Hue ← IR}^{PPG} = {\sum }_{\frac{t}{2}} {\sum }_{λ} {\sum }_{\vec{x} , \vec{y}} \hat{P} ( λ ) {v}_{AC} ( \vec{x} , \vec{y} , t ) {b}_{A C} ( t )$$

$${I}_{Hue ← Red}^{PPG} = {\sum }_{\frac{t}{2}} {\sum }_{λ} {\sum }_{\vec{x} , \vec{y}} \hat{P} ( λ ) {v}_{AC} ( \vec{x} , \vec{y} , t ) {b}_{A C} ( t )$$

The ratio of DC and AC components from I_{HueΘRed}^{PPG} yields I_{Hue←Red}^{PPG}^{_{AC/DC}}. Similarly, the ratio of DC and AC components from I_{Hue←IR}^{PPG} yields I_{Hue←IR}^{PPG}^{_{AC/DC}}. According to an embodiment of the present disclosure, SpO₂ is computed using R_{Hue←Red,Hue←IR}:

$${R}_{Hue ← Red , Hue ← IR} = \frac{{I}_{Hue ← Red}^{{PPG}_{AC \text{/} DC}}}{{I}_{Hue ← IR}^{{PPG}_{AC \text{/} DC}}}$$

$${R}_{Hue ← Red , Hue ← IR} = \frac{{I}_{Hue ← IR}^{{PPG}_{AC}}}{{I}_{Hue ← Red}^{{PPG}_{AC}}} \times \frac{highpass ( {I}_{Hue ← Red}^{{PPG}_{D C}} )}{highpass ( {I}_{Hue ← IR}^{{PPG}_{D C}} )} = \frac{{\sum }_{\frac{t}{2}} {\sum }_{Hue ← IR} {\sum }_{\vec{x} + \vec{y}} \hat{P} ( λ ) {v}_{AC} ( \vec{x} , \vec{y} , \frac{t}{2} ) {b}_{AC} ( λ , \frac{t}{2} )}{{\sum }_{\frac{t}{2}} {\sum }_{Hue ← Red} {\sum }_{\vec{x} + \vec{y}} \hat{P} ( λ ) {v}_{AC} ( \vec{x} , \vec{y} , \frac{t}{2} ) {b}_{AC} ( λ , \frac{t}{2} )} \times \frac{highpass ( {\sum }_{\frac{t}{2}} {\sum }_{Hue ← Red} {\sum }_{\vec{x} + \vec{y}} \hat{P} ( λ ) {v}_{AC} ( \vec{x} , \vec{y} , \frac{t}{2} ) {b}_{AC} ( λ , \frac{t}{2} ) )}{highpass ( {\sum }_{\frac{t}{2}} {\sum }_{Hue ← IR} {\sum }_{\vec{x} + \vec{y}} \hat{P} ( λ ) {v}_{AC} ( \vec{x} , \vec{y} , \frac{t}{2} ) {b}_{AC} ( λ , \frac{t}{2} ) )}$$

When using conventional photodiode-based pulse oximetry, PPGs based on these variables I_{λ}_{ₙ}^{PPG}^{_{AC/DC}} are independent of the intensity of incident light I(\vec{x},\vec{y},t).

$${I}_{{λ}_{n}}^{{PPG}_{AC \text{/} DC}} = \frac{{\sum }_{\frac{t}{2}} {v}_{DC} ( \vec{x} , \vec{y} , \frac{t}{2} ) {\sum }_{{λ}_{n}} {\sum }_{\vec{x} + \vec{y}} \hat{P} ( λ ) h ( λ , \vec{x} , \vec{y} , \frac{t}{2} ) {b}_{DC} ( λ , \frac{t}{2} )}{{\sum }_{\frac{t}{2}} {v}_{A C} ( \vec{x} , \vec{y} , \frac{t}{2} ) {\sum }_{{λ}_{n}} {\sum }_{\vec{x} + \vec{y}} \hat{P} ( λ ) h ( λ , \vec{x} , \vec{y} , \frac{t}{2} ) {b}_{A C} ( λ , \frac{t}{2} )}$$

However, I_{λ}_{ₙ}^{PPG}^{_{AC/DC}} is still dependent on the volume of static and pulsatile blood:

$$\frac{{v}_{DC} ( \vec{x} , \vec{y} , t )}{{v}_{A C} ( \vec{x} , \vec{y} , t )} .$$

This dependence can be reduced by taking the ratio of ratios at two different wavelengths λₙ.

$${R}_{{λ}_{1} {λ}_{2}} = \frac{{I}_{{λ}_{1}}^{{PPG}_{AC \text{/} DC}}}{{I}_{{λ}_{2}}^{{PPG}_{AC \text{/} DC}}}$$

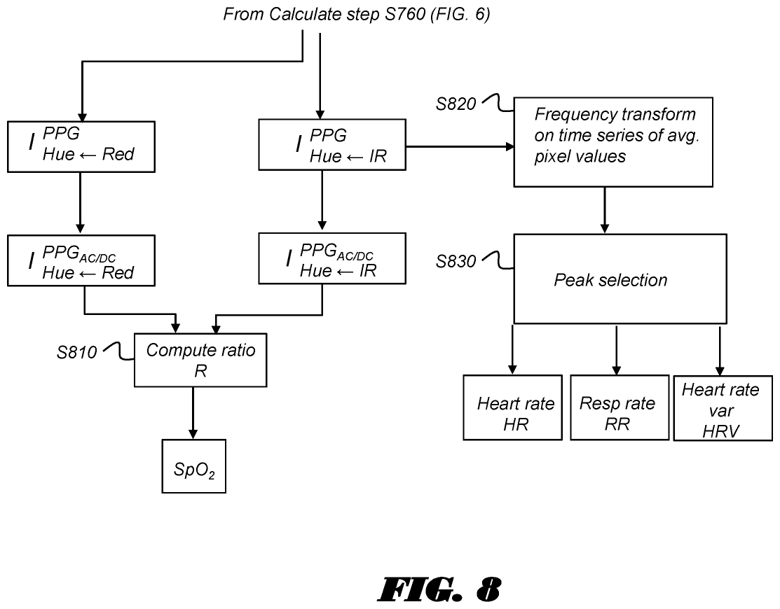

The logic flow diagram of FIG. 8 shows a sequence for continuing calculations that obtain HR and RR values and compute the SpO₂ value. Color PPGs I_{Hue←Red}^{PPG} and I_{Hue←IR}^{PPG} are obtained from preceding calculation (FIG. 6). Then, the needed AC/DC ratios are computed for Hue values in Red and IR ranges, as described previously. A computation step S810 computes the ratio of ratios R that obtains the SpO₂ measurement. For HR and RR computation, a transform step S820 executes a Fourier transform, or other suitable transform or method, for defining frequency and peak values of interest. A peak identification step S830 then selects peak values used for HR and RR computation.

The timing diagram of FIG. 9 shows synchronization of IR and Red illumination used to acquire PPG data used for SpO₂ measurement. According to an embodiment of the present disclosure, the Red and IR illumination sources are alternately energized every half-cycle, indicated as time t′. Time t′ can be, for example, 33.3 msec. Sampling occurs once every interval t′, alternating between Red and IR emission wavelengths. As a result, within time interval t, there are t/2 Red samples and t/2 IR samples, as described in the sequence given above.

The invention has been described in detail with particular reference to certain preferred embodiments thereof, but it will be understood that variations and modifications can be effected within the scope of the invention as described above, and as noted in the appended claims, by a person of ordinary skill in the art without departing from the scope of the invention.

1. A method for photoplethysmography measurement comprising:
a) coupling a color camera in optical contact against a skin surface of a subject;
b) coupling at least a first solid-state illumination source in optical contact against the skin surface, wherein the first solid-state illumination source has a first wavelength range with a first bandwidth that exceeds 50 nm;
c) energizing the illumination source and color camera over a predetermined time interval to acquire a first sequence of a plurality of image frames from the skin surface;
d) computing a set of hue values from each of the acquired sequence of image frames and generating photoplethysmography data according to periodic changes in an average hue per frame computation; and
e) presenting the generated photoplethysmography data on a display.

2. The method of claim 1 wherein values for extinction coefficients of Hb and HbO₂ at the skin surface are unequal at each wavelength within the first wavelength range.

3. The method of claim 1 further comprising:
f) coupling a second solid-state illumination source in optical contact against the skin surface, wherein the second illumination source has a second wavelength range that lies outside the first wavelength range and wherein, over the full second wavelength range, extinction coefficients for hemoglobin exceed extinction coefficients for oxyhemoglobin;
g) alternately energizing the first and second illumination sources over a predetermined time interval and energizing the color camera to acquire both the first sequence of image frames and a second sequence of a plurality of image frames from the skin surface over the same time interval; and
h) computing periodic changes in hue values computed from the first and sequence sequences of image frames and generating first and second sets of photoplethysmography data according to averaged hue per frame.

4. The method of claim 3 wherein presenting the photoplethysmography data further comprises displaying photoplethysmography data from either the first or the second sequence of image frames according to a signal-to-noise ratio.

5. The method of claim 1 wherein optical contact is provided by a light guide.

6. The method of claim 5 wherein the light guide is formed of an optical polycarbonate.

7. The method of claim 1 wherein optical contact is further provided by a material applied to the skin.

8. The method of claim 1 further comprising wirelessly transmitting the generated PPG data to a processor.

9. The method of claim 5 wherein the light guide is formed of an optical liquid silicone rubber.

10. The method of claim 1 further comprising computing a heart rate of the subject according to the generated photoplethysmography data.

11. The method of claim 1 further comprising computing a respiration rate of the subject according to the generated photoplethysmography data.

12. The method of claim 1 wherein the skin surface is along a sternum.

13. The method of claim 3 further comprising computing a hemoglobin saturation value SpO₂ according to ratios of the first and second sets of photoplethysmography data.

14. A method for photoplethysmography acquisition comprising:
a) coupling a color camera in optical contact against a skin surface of a subject;
b) coupling a first solid-state light source and a second solid-state light source in optical contact against the skin surface, wherein the first solid-state light source has a first wavelength range and the second light source has a second wavelength range,
wherein the first and second wavelength ranges are non-overlapping,
and wherein, over both the first and second wavelength ranges, values for extinction coefficients of hemoglobin Hb and oxygenated hemoglobin HbO₂ at the skin surface are unequal at each wavelength;
c) alternately energizing the first light source and second light source over a predetermined time interval;
d) acquiring, at the color camera, a first sequence of images from the skin surface using light of the first wavelength range and a second sequence of images from the skin surface using light of the second wavelength range;
e) for the first sequence of images, computing periodic changes in hue over the predetermined time interval and generating a first set of photoplethysmography data according to the hue computation;
f) for the second sequence of images, computing periodic changes in hue over the predetermined time interval and generating a second set of photoplethysmography data according to the hue computation;
g) computing a heart rate and a respiratory rate from either the first or second set of photoplethysmography data; and
h) computing a hemoglobin saturation value SpO₂ according to ratios computed using the first and second sets of photoplethysmography data.

15. The method of claim 14 wherein optical contact is provided by a light guide.

16. The method of claim 15 wherein the light guide is formed of an optical polycarbonate.

17. The method of claim 14 wherein optical contact is provided by a material applied to the skin.

18. The method of claim 14 wherein, over the first wavelength range, the extinction coefficient of Hb exceeds the extinction ration of HbO₂.
